SEQUENCE LISTING (4 sequences)

SEQ ID NO: 1 - protein, 12 aa, Homo sapiens
HKNQKDDVAQTD

SEQ ID NO: 2 - protein, 18 aa, Homo sapiens
MAEDKSKRDSIEMSMKGC

SEQ ID NO: 3 - protein, 15 aa, Mus musculus
CHLQMLEVVPEKDPE

SEQ ID NO: 4 - protein, 15 aa, Mus musculus
PSPLPITDKPLKTQC

(19) United States
(12) Patent Application Publication
(10) Pub. No.: US 2009/0041719 A1
(43) Pub. Date: Feb. 12, 2009

(54) Method for screening therapeutic agent for glomerular disorder

(76) Inventor: Michio Ishibashi, Sakai-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/987,874

(22) Filed: Dec. 5, 2007

Related U.S. Application Data
(63) Continuation-in-part of application No. PCT/JP2007/061573, filed on Jun. 7, 2007.

(30) Foreign Application Priority Data
Jun. 9, 2006 (JP) 2006-161569

(51) Int. Cl. A61K 38/21; C12Q 1/02; A61P 13/12

(52) U.S. Cl. 424/85.4; 435/29

(57) ABSTRACT

The present invention relates to a method for screening a compound for preventing, ameliorating or treating glomerular lesions or disease of kidney, characterized by assaying the regulating activity of compound on 4F2hc expression shown by contacting human peripheral blood mononuclear cells or monocytes or an established line of human cultured cells having a nature of human macrophages with macrophage-activating substances. The screening method is useful for screening compounds capable of preventing, ameliorating or treating glomerular lesions or disease of kidney.

### TECHNICAL FIELD

The present invention relates to a method for screening a therapeutic agent for glomerular lesions or disease of kidney, and more particularly to a method for screening a compound which prevents, ameliorates, or treats glomerular lesions or disease of kidney, by assaying 4F2hc expressed on human peripheral blood mononuclear cells or human monocytes, or an established line of human cultured cells having a nature of human macrophages.

### BACKGROUND ART

It has been suggested that upon repair and regeneration after organ or tissue damage, there is a host-intrinsic autoimmune cell response involved in nervous tissue, skin wounds, renal tubular injuries, and the like (Renal Failure (1996); 18:355-375, Immunology Today (2000); 21:265-269, and J Nephrology (2003); 16:186-195). In addition, it is known that compounds which induce and promote CD11b⁺ CD2⁺ macrophages and regulatory CD2⁻ CD4⁺ T lymphocytes selectively repair glomerular lesions or disease of kidney (International Publication WO 2004/024185 pamphlet), and that compounds which inhibit the induction of effecter macrophages have effects of improving glomerular lesions in the kidney (International Publication WO 01/72730 pamphlet).

It has been suggested that, in order to repair tissue after organ damage, repair is required at the DNA level, cell level, and organ-structure level. In the cell level, cell growth and protein synthesis including growth and differentiation of precursor cells existing in an organ are required, and in structural repair and remodeling, damaged tissue is repaired corresponding to the level of injury (J Nephrology (2003); 16:186-195).

In order to stimulate protein synthesis of damaged tissue cells corresponding to injury thereby leading to repair or regeneration thereof, it is expedient that cells having a repair and regeneration action have molecules which selectively adhere to and fuse to the tissue cells of lesions and express molecules promoting protein synthesis at the same time.

Fusion-regulatory protein (FRP)-1, CD98, and 4F2hc (4F2 heavy chain) are the same molecule, which are also referred to as “FRP-1/CD98/4F2hc molecule”. This molecule is known to express on T cells and macrophages upon immunostimulation or viral infection and to promote fusion to form polykaryocytes upon viral infection (Critical Review in Immunology (2000); 20: 167-196). The molecule is involved in various cell functions such as functions associated with P1 integrin, hematopoiesis, apoptosis, lymphocyte growth, functions of B and T lymphocyte, cell fusion to osteoclastogenesis, mutagenesis, and cell fusion after viral infection. The above-mentioned molecule (FRP-1/CD98/4F2hc molecule) constitutes heavy chains (H chains) of heteromeric amino acid transporters (hereinafter, abbreviated as HAT) of amino acid transporters (Current Drug Metabolism (2001); 2: 339-354, and Physiology (2005); 20: 112-124). It is known that these heteromeric amino acid transporters are expressed on human macrophages also (Am J Physiol Cell Physiol (2001); C1964-C1970). This heteromeric amino acid transporter has little selectivity about amino-acid transport, and non-specifically promotes protein synthesis.

It is also known that some kinds of compounds (such as Cynaropicrin) inhibit the CD98 molecule thereby inhibiting cell agglutination (Critical Review in Immunology (2000); 20: 167-196, and Biochemical and Biophysical Research Communications (2004); 313: 954-961).

However, compounds which induce cells involved in repair and regeneration and expressing heteromeric amino acid transporters by regulating the expression of FRP-1/CD98/4F2hc molecules are not known, and there is no screening method to identify a compound having the above-mentioned functions in a test tube.

### DISCLOSURE OF THE INVENTION

### Problems to be Solved by the Invention

The present invention provides a method for screening a therapeutic agent for glomerular disease of kidney.

### SUMMARY OF THE INVENTION

### Means for Solving the Problems

The present inventor has found that mononuclear cells expressing Asc-1 molecule, one of heteromeric amino acid transporters, on renal glomerular lesions of an animal that has been treated with a known compound having an action of selectively reducing and repairing the glomerular lesions (International Publications WO 2004/024185 and 01/72730 pamphlets) localize more significantly on sites of glomerular lesions compared to a control animal, and that the glomerular lesion of the former animal has been significantly reduced.

The present inventor has further found that, in the case where human peripheral blood mononuclear cells are stimulated and cultured with lipopolysaccharide (LPS), or where human peripheral blood mononuclear cells or human monocytes, or an established line of human cultured cells having the nature of human macrophages (hereinafter, sometimes referred to as “human cultured cell line”) is stimulated and cultured with interferon, when a known compound which selectively inhibits and repairs glomerular lesions (International Publication WO 2004/024185 and 01/72730 pamphlets) is added thereto before culture, the resulting system has an action of selectively regulating the expression of FRP-1/CD98/4F2hc molecule, the H (heavy) chain of a heteromeric amino acid transporter.

That is, the expression of a nerve-cell-derived Asc-1 (asc-type amino acid transporter 1), one of heteromeric amino acid transporters, by the mononuclear cells or the established line of human cultured cells means that the FRP-1/CD98/4F2hc molecules, which are H chain molecules, are activated, whereby the glomeruli adhere and fuse with affinity to damaged cells in the glomerular lesions, in connection with a property in which the glomeruli have also molecules of nerve cells, and that the Asc-1 can widely provide amino acids which are necessary for damaged organ tissue cells and promote protein synthesis to repair the damaged cells.

Therefore, from the facts that a compound known to selectively inhibit and repair renal glomerular lesions selectively induce mononuclear cells which can express Asc-1 to sites of glomerular lesions and that the known compound has an action of selectively regulating the expression of the FRP-1/CD98/4F2hc molecules that are the H chain of the heteromeric amino acid transporter when added to human peripheral blood mononuclear cells or the established line of human cultured cells at the start of culture thereof stimulated by macrophage-activating substances such as lipopolysaccharide (LPS) or interferon, the inventor has found that screening methods to readily search and identify corresponding compounds can be provided.

The present inventor has further repeated studies based on the above-mentioned findings, and completed the present invention.

That is, the present invention relates to the following methods:

(1) a method for screening a compound for preventing, ameliorating or treating glomerular lesions or disease of kidney, characterized by assaying the regulating action shown by the test compound on the expression of 4F2hc caused by contacting human peripheral blood mononuclear cells or human monocytes, or an established line of human cultured cells having a nature of human macrophages with a macrophage-activating substance;
(2) the screening method of the above-mentioned (1), wherein the assay of the regulating action shown by the test compound is performed in a manner in which the human peripheral blood mononuclear cells or human monocytes, or the established line of human cultured cells having a nature of human macrophages is cultured in the presence of the macrophage-activating substance and the test compound, and the amount of 4F2hc expressed on the mononuclear cells or the human monocytes, or the established macrophage line of human cultured cells is compared to the amount of 4F2hc expressed when they are cultured in the absence of the test compound;
(3) the screening method of the above-mentioned (2), wherein the human peripheral blood mononuclear cells are cultured in the presence of human AB serum, or the established line of human cultured cells having a nature of human monocytes or human macrophages are cultured in the presence of fetal bovine serum;
(4) the screening method of the above-mentioned (2), wherein the amount of 4F2hc expressed on the mononuclear cell or the established line of human cultured cells having a nature of human monocytes or human macrophages is calculated by FACScan analysis;
(5) the screening method of the above-mentioned (1), wherein the macrophage-activating substance is interferon or lipopolysaccharides;
(6) the screening method of the above-mentioned (1), wherein the established line of human cultured cells having a nature of human monocytes or human macrophages is Human leukemia culture cell line THP-1;
(7) a method for screening a compound capable of preventing, ameliorating or treating glomerular lesions in the kidney comprising the steps of: (a) culturing human peripheral blood mononuclear cells in the presence of lipopolysaccharides and a test compound in human AB serum; (b) recovering the human peripheral blood mononuclear cells from the resulting culture fluid; (c) reacting antibodies of 4F2hc with the recovered human peripheral blood mononuclear cells; (d) calculating the amount of 4F2hc by FACScan analysis; and (e) comparing the amount of 4F2hc with the amount of 4F2hc cultured in the absence of the test compound to detect increase or decrease of the amount of 4F2hc;
(8) a method for screening a compound capable of preventing, ameliorating or treating glomerular lesions or disease of kidney comprising the steps of: (a) culturing an established line of human cultured cells having a nature of human macrophages in the presence of (i) lipopolysaccharides or interferon and (ii) a test compound in fetal bovine serum; (b) recovering the established line of human cultured cells having a nature of human macrophages from the resulting culture fluid; (c) reacting antibodies of 4F2hc with the ed established line of human cultured cells; (d) calculating the amount of 4F2hc by FACScan analysis; and (e) comparing the amount of 4F2hc with the amount of 4F2hc cultured in the absence of the test compound to detect increase or decrease of the amount of 4F2hc;
(9) a pharmaceutical composition which prevents, ameliorates or treats glomerular lesions or disease of kidney, containing a combination of (i) a compound having an action of regulating the expression of 4F2hc caused by contacting human peripheral blood mononuclear cells or human monocytes, or an established line of human cultured cells having a nature of human macrophages with a macrophage-activating substance (ii) and interferon;
(10) the pharmaceutical composition of the above-mentioned (9), wherein the compound is a compound selected by the screening method of the above-mentioned (1);
(11) the pharmaceutical composition of the above-mentioned (9), wherein the compound is 2-fluoro-5-oxotetrahydrofuran-2-carboxylic benzyl ester or 1-(4-fluorophenoxy)-3-oxo-1,3-dihydro-isobenzofuran-1-carboxylic acid;
(12) the pharmaceutical composition of the above-mentioned (9), wherein the pharmaceutical composition which prevents, ameliorates or treats glomerular lesions or disease of kidney is a combination drug;
(13) the pharmaceutical composition of the above-mentioned (8), wherein the pharmaceutical composition which prevents, ameliorates or treats glomerular lesions or disease of kidney is a kit comprising (i) a medicinal agent containing a compound having an action of regulating the expression of 4F2hc induced by contacting human peripheral blood mononuclear cells or human monocytes, or an established line of human cultured cells having a nature of human macrophages with a macrophage-activating substance and (ii) a medicinal agent containing interferon; and
(14) a method for preventing, ameliorating or treating glomerular lesions or disease of kidney including the step of administrating (i) a compound which prevents, ameliorates or treats glomerular lesions or disease of kidney and which is selected by the screening method of the above-mentioned (1) and (ii) interferon to a patient in need of such prevention, amelioration or treatment of glomerular lesions or disease of kidney.

### Effects of the Invention

According to the screening method of the invention, compounds capable of preventing, ameliorating, or treating glomerular lesions in the kidney can be readily searched for and identified.

### BEST MODE FOR CARRYING OUT THE INVENTION

The method for screening compounds capable of preventing, ameliorating, or treating glomerular lesions or disease of kidney according to the invention is characterized by assaying the regulating action shown by the test compound on the expression of 4F2hc caused by contacting human peripheral blood mononuclear cells or human monocytes, or an established line of human cultured cells having a nature of human macrophages with a macrophage-activating substance.

The assay of the regulating action shown by the test compound is performed in a manner in which the human peripheral blood mononuclear cells or human monocytes, or the established line of human cultured cells having a nature of human macrophages is cultured in the presence of the macrophage-activating substance and the test compound, and the amount of 4F2hc expressed on the mononuclear cells, or monocytes, or the established line of human cultured cells is compared to the amount of 4F2hc expressed when they are cultured in the absence of the test compound.

The human peripheral blood mononuclear cells (which can be abbreviated as “PBMCs”) used in the present screening method can be obtained from human peripheral blood by methods known in the art. For example, as a method for separating mononuclear cells from human peripheral blood, a centrifugal separation method using 5.7% w/v of Ficoll 400 and Ficoll-Paque (registered trademark, made by Pharmacia Fine Chemicals) which is an aqueous solution of 9.0% w/v sodium diatrizoate with adjusted specific gravity of 1.077 g/mL, may be mentioned. More specifically, the above-mentioned method comprises: (a) a step of placing a predetermined amount of Ficoll-Paque at the bottom of a test tube; (b) a step of carefully placing a blood sample as it is or diluted on the Ficoll-Paque with a pipette; (c) a step of centrifuging the Ficoll-Paque blood preparation made in the step (b) at about 400 to 500×g for about 30 to 40 minutes so that a blood component having specific gravity heavier than that of Ficoll-Paque goes into or passes through the Ficoll-Paque; and (d) a step of collecting the layer of mononuclear cells separated on top of the Ficoll-Paque with a pipette.

The established line of human cultured cell having a nature of human macrophage refers to the one which responds to the stimulus of a macrophage-activating substance similar to a normal human monocyte or macrophage. Examples of such an established line of human cultured cells may include, for example, a human leukemia culture cell line, and the like, and specifically include the human leukemia culture cell line THP-1. The human leukemia culture cell line THP-1 to use can be purchased from Health Science Research Resources Bank (Sennan-Shi, OSAKA). The THP-1 is a culture cell line having a macrophage-like cell activity, which is established from the one derived from the peripheral blood of a patient with human acute monocytic leukemia.

On the other hand, the macrophage-activating substance used in the screening method of the invention is not particularly limited so long as the substances expresses a heteromeric amino acid transporter, that is, an Asc-1 molecule, LAT1 (L-type amino acid transporter 1), LAT2 (L-type amino acid transporter 2) (these have a FRP-1/CD98/4F2hc molecule on their H chain), or the like, on a human peripheral blood mononuclear cell (PBMC) or a human cultured cell line, and is preferably lipopolysaccharides (LPS) or interferon. The interferon may include interferon α, interferon β, interferon γ, and the like, and is preferably interferon γ.

The lipopolysaccharides can be prepared by methods known in the art. Examples of them include a method in which lipopolysaccharides are extracted from a microbe and toxic substances are removed therefrom, if desired. The extraction from the microbe may include, for example, heat-phenol method extraction (Westphal & Jann., Methods Carbohydr. Chem. 5, 83-89 (1965)), a method in which the microbe is treated with proteinase K in the presence of sodium lauryl sulfate (SDS), and the like. Chemically synthesized ones may be used, or commercially available ones may suitably be used. In the present invention, a solution, which is obtained by using a suitable solvent, preferably RPMI 1640 liquid, has a concentration of about 60 to 100 μg/mL, preferably about 70 to 90 μg/mL, more preferably a high concentration of about 80 μg/mL. As the lipopolysaccharides, commercially available products (such as PLS derived from Escherichia coli made by Sigma, catalog number: L2654) can be preferably used. Meanwhile, as the interferon, human recombinant interferon produced by genetic engineering techniques can be preferably used.

As the medium used in the culture of the human peripheral blood mononuclear cells or monocytes or the established line of human cultured cells, RPMI medium is preferable. This RPMI medium is described in Goding, J. W. (1980) J. Immunol. Methods 39, 285, JAMA 199 (1957) 519. A commercially available product (made by Sigma) may also be used.

At about 37° C., the human peripheral blood mononuclear cells can be preferably cultured in the presence of human AB serum, and the established line of human cultured cells can be preferably cultured in the presence of fetal bovine serum. All of the “test compound,” the “lipopolysaccharides or interferon,” the “human AB serum or fetal bovine serum,” and the “human peripheral blood mononuclear cells or the established line of human cultured cells”, or any combination of two kinds thereof may be previously mixed and then added to the above-mentioned medium, or each of them may be independently added to the medium. The human peripheral blood mononuclear cells or monocytes or the established line of human cultured cells is cultured for usually 2 to 14 days, preferably 6 to 10 days, preferably at a temperature of about 37° C. Preferably, the culture is carried out under about 5% CO₂ conditions.

After the culture, the human peripheral blood mononuclear cells or the human cultured cells are recovered, FRP-1/CD98/4F2hc molecules expressed on the mononuclear cells or monocytes or the established line of human cultured cells are reacted with antibodies against the molecules, and the antibody-bound human peripheral blood mononuclear cells or established line of human cultured cells are analyzed by, for example, FACScan, whereby the amount of the FRP-1/CD98/4F2hc molecules expressed on the mononuclear cells or monocytes or the established line of human cultured cells can be calculated.

The antibody against the FRP-1/CD98/4F2hc molecule may include monoclonal antibodies and polyclonal antibodies. The monoclonal antibodies can be prepared, for example, in accordance with a method described in J Virology (1992); 66: 5999-6007 as follows: that is, a BALB/c mouse is immunized with the human epithelial cultured cell line FL, the spleen of the mouse is collected, and the cell fusion between a cell of the spleen and an SP2/0-AG-14 myeloma cell is performed. As the screening to detect hybridoma cells, a fusion-enhancing assay using FL or Hela cells infected with Newcastle disease virus is used. By this means, hybridoma cells capable of producing mouse anti-human monoclonal antibody 4-5-1 or 6-1-3 can be detected. The hybridoma cells are statically cultured in MEM culture fluid supplemented with 5% fetal bovine serum. The culture supernatant can be used at its stock concentration in FACScan. Similarly, HBJ-127 hybridoma cells obtained using the human bladder cancer cultured line T24 as immunogen (see Jpn J Cancer Res. (Gann) (1985); 76: 336), are statically cultured in MEM culture fluid supplemented with 5% fetal bovine serum. The culture supernatant can be used at its stock concentration in FACScan. The natures of the above-mentioned monoclonal antibody 4-5-1, 6-1-3, and HBJ-127 are described in Critical Reviews in Immunology, volume 20/issue 3, 2000, 167-196. The polyclonal antibodies against the FRP-1/CD98/4F2hc molecule can be obtained by synthesizing a peptide corresponding to an amino acid residue of 164-175 of human 4F2hc (HKNQKDDVAQTD (SEQ ID NO: 1)), introducing a cysteine residue at the C-terminal, coupling it with KLH (keyhole-limpet hemocyanin), immunizing a rabbit therewith, and purifying it using an affinity column with the same peptide (164-175 of human 4F2hc) bound thereto.

The FACScan is performed by liberating the monoclonal antibodies or polyclonal antibodies on the recovered human peripheral blood mononuclear cells or established line of human cultured cells; then liberating fluorescent antibodies which are secondary antibodies (such as FITC-goat IgG); putting the human peripheral blood mononuclear cells or the established line of human cultured cells stained with fluorescent antibodies in flowing liquid current; getting it go through the focus of laser beam; and measuring the fluorescence emitted by each cell in order to measure the amount of the FRP-1/CD98/4F2hc molecules expressed on the human peripheral blood mononuclear cells or the established line of human cultured cells. In addition, according to FACScan, the protein in cytoplasm can be simultaneously measured by making a hole on the cell membrane. The procedure to simultaneously measure the protein in cytoplasm will be described below. First, cells are immobilized. That is, as described above, an about 4% formaldehyde solution is added to the recovered human peripheral blood mononuclear cells or the established line of human cultured cells, which is left stand still at about 37° C. for about 10 minutes to immobilize the cells (Cytometry Part A (2003); 55A: 61-70). For this cell immobilization, for example, the BD Cytofix/Cytoperm™ liquid in the commercially available cell immobilization/cell membrane infiltration kit (trade name: BD Cytofix/Cytoperm™ Kit, catalog No. 554714, BD Bioscience Corporation) can be used. Next, permeabilization is performed so that the antibodies can pass through the cell membrane. That is, an about 0.1% saponin solution is added to the immobilized human peripheral blood mononuclear cells or the immobilized established line of human cultured cells, which is left stand still at about 4° C. for about 30 minutes. For this permeabilization, for example, the BD Perm/Wash™ staining buffer including 0.1% saponin in the above-mentioned commercially available cell immobilization/cell membrane infiltration kit can be used. And then, immunostaining is performed. That is, rabbit anti-human 4F2hc polyclonal antibodies which are primary antibodies are reacted with the permeabilized human peripheral blood mononuclear cells or the permeabilized cells of the established line of human cultured cells. After that, as secondary antibodies, fluorescence-labeled antibodies (such as goat FITC-anti-rabbit immunoglobulin) are reacted therewith. The above-mentioned human peripheral blood mononuclear cells or the established line of human cultured cells stained with fluorescence-labeled antibodies is put in flowing liquid current, passed through the focus of laser beam, and the fluorescence emitted by each cell is measured in order to measure the an amount of 4F2hc molecules expressed on the human peripheral blood mononuclear cells or monocytes or the established line of human cultured cells.

The amount of the FRP-1/CD98/4F2hc molecules as measured above is compared to the amount of 4F2hc expressed in the absence of the test compound and the increase or the decrease of the FRP-1/CD98/4F2hc molecules is detected, whereby the regulating action of the test compound can be assayed against the expression of FRP-1/CD98/4F2hc molecules. The identification and selection of a compound which increases a lowered amount of 4F2hc, or a compound which decreases an elevated amount of FRP-1/CD98/4F2hc molecules, can lead to a screening of a compound capable of preventing, ameliorating or treating glomerular lesions or disease of kidney.

Another embodiment of the invention is a pharmaceutical composition which prevents, ameliorates or treats glomerular lesions or disease of kidney, containing a combination of a compound (hereinafter which can be referred to as “effective component compound”) having an action of regulating the expression of 4F2hc caused by contacting human peripheral blood mononuclear cells or human monocytes, or an established line of human cultured cells having a nature of human macrophages with a macrophage-activating substance, and interferon. Using the above-mentioned effective component compound and the interferon in combination can enhance the action of preventing, ameliorating or treating glomerular lesions or disease of kidney compared to using the compound alone.

The examples of the effective component compounds may include compounds which prevent, ameliorate or treat glomerular lesions or disease of kidney, which are selected by the above-mentioned screening methods. Such compounds may include compounds described in International Publication WO 01/72730 pamphlet such as compounds represented by the following formulae (I) and (II):

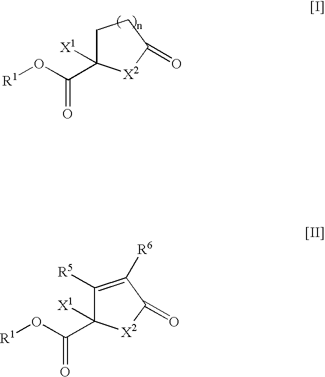

wherein the symbols have the same meanings as described in the International Publication WO 01/72730 pamphlet.)

Specifically, 2-fluoro-5-oxotetrahydrofuran-2-carboxylic benzyl ester,

1-(4-fluoro-phenoxy)-3-oxo-1,3-dihydro-isobenzofuran-1-carb oxylic acid, and the like can be cited. Also, as the above-mentioned effective component compound, the compound represented by the following formula (3-2) can be cited.

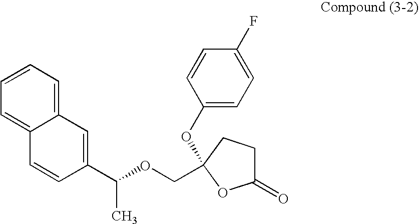

The pharmaceutical composition of the present invention may be a combination drug of the above-mentioned effective component compounds and interferon, or may be a kit comprising medicinal agents, each of which contains either one or the other component. When the composition is the kit, the medicinal agent containing the effective component compound and the medicinal agent containing interferon may be separately administered or may be concurrently administered. The molar ratio of the effective component compound and the interferon is usually from 1:0.001 to 1:0.5, preferably from 1:0.02 to 1:0.1.

### EXAMPLES

Hereinafter, the present invention will be described in more detail in reference to experimental examples, but the invention is not limited thereto. The test compounds used in the examples are the compound (6-2) and the compound (3-2) known to selectively inhibit glomerular lesions, and the compound (4-2) and the compound (7-3) known to selectively inhibit renal tubulointerstitial lesions. They are respected by the following structure formulae (see International Publication WO 2004/024185 and 01/72730 pamphlets):

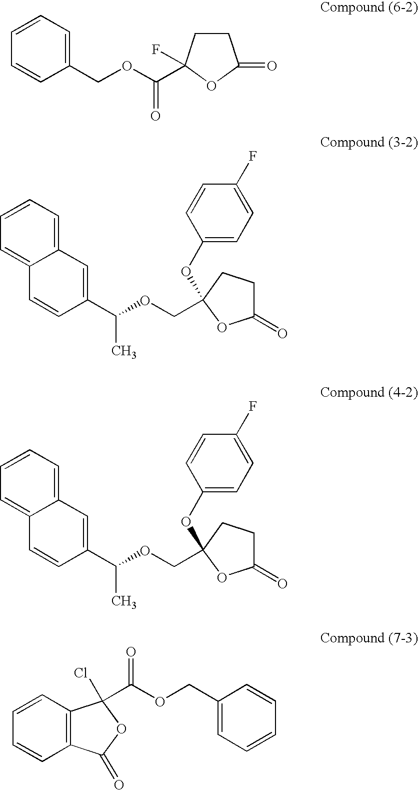

It is known that among the above-mentioned compounds, the compound (6-2) and the compound (3-2) selectively inhibit the glomerular lesions, and the compound (4-2) and the compound (7-3) selectively inhibit the renal tubulointerstitial lesions (International Publication WO 2004/024185 and 01/72730 pamphlets).

### Experimental Example 1

### Experimental Procedure

### 1) Separation of Human Peripheral Blood Mononuclear Cells (Referred to as PBMCs):

A syringe was charged with a small amount of heparin from normal human peripheral blood, followed by collection of about 30 ml of blood, to which an equal amount of 0.9% physiologic saline (supplemented with 1 mM EDTA) was added immediately and mixed therewith. In a 50 ml tube, 12 ml of HISTOPAQUE (made by Sigam-Aldrich Co., Cat No. 1119-1) and 10 ml of Ficoll-Paque Plus (made by Amersham Bioscience) were layered, onto which the blood was laid gently. The tube was centrifuged at 400 g at room temperature for 20 minutes. About 8 ml of a plasma component was collected and passed through a 0.2 μm Millipore filter. The mononuclear cell fraction containing lymphocytes plus monocytes was collected, a sufficient amount of cooled PBS (Ca⁺⁺) was added thereto and mixed, and the mixture was subjected to centrifugal washing at 250 g at 4° C. for 10 minutes. The supernatant was thrown away, and the above-mentioned procedure was repeated twice. About 8 ml of the collected plasma was added to pellets. After mixing them, the mixture was left stand still at 37° C. for 10 minutes. The mixture was subjected to centrifugal washing at 250 g at 4° C. for 10 minutes. RPMI 1640 with no serum (2 mM L glutamine, 5 μg/ml gentamicin, hereinafter referred to as “culture fluid RPMI”) was added to the pellets, and centrifugal washing was performed in the same manner as described above. The final number of cells of PBMC was adjusted to 2×10⁶/ml.

### 2) Preparation of Lipopolysaccharides (LPS):

1 mg of LPS derived from Escherichia coli (Sigma, catalog number L2654) was diluted with 5 ml of the culture fluid RPMI, which was passed through a 0.2 μl Millipore filter, which was used as LPS.

### 3) Dilution of Test Compound:

The test compound was dissolved in a stock solution of sterilized DMSO (dimethyl sulfoxide), which was diluted with the culture fluid RPMI to adjust the final DMSO concentration to 10⁻⁵ (v/v), and the activity was studied at a test compound concentration of 100 nM or less.

### 4) Culture:

To each well of a microplate (Falcon 3046) were added 1000 μl of PBMC solution, the same amount of LPS, 250 μl of the test compound solution, and 250 μl of human AB serum, which was cultured at 37° C. under 5% CO₂-95% air for 6 days.

### 5) Retrieval of Cultured PBMCs:

After the completion of culture, PBMCs including attached cells were recovered using a rubber. The culture fluid was washed once with Hanks solution (supplemented with 5 μl/ml gentamicin) with no serum, and the concentration (cell number) was adjusted with the same solution to 4×10⁶/ml.

6) Preparation of Monoclonal Antibodies against FRP-1/CD98/4F2hc (Mouse Anti-human Monoclonal Antibodies 4-5-1, 6-1-3, and HBJ-127):

Mouse anti-human monoclonal antibodies 4-5-1 and 6-1-3 were prepared in accordance with a method described in J Virology (1992); 66: 5999-6007. That is, BALB/c mouse was immunized with the human epithelial culture cell line FL to collect the spleen of the mouse, and cell fusion with SP^{2/0}-AG-14 myeloma cell was performed. For screening to detect hybridoma cells, fusion-enhancing assay using FL cells or Hela cells infected with Newcastle disease virus, was employed, the hybridoma cells producing the mouse anti-human monoclonal antibodies 4-5-1 or 6-1-3 were detected thereby.

The hybridoma cells were statically cultured in MEM culture fluid supplemented with 5% fetal bovine serum. The culture supernatant was used in FACScan at its stock concentration.

Similarly, HBJ-127 hybridoma cells obtained using the human bladder cancer culture lines T 24 as an immunogen (see Jpn J Cancer Res. (Gann) (1985); 76: 336), were statically cultured in MEM culture fluid supplemented with 5% fetal bovine serum. The culture supernatant was used in FACScan at its stock concentration.

The natures of the mouse anti-human monoclonal antibodies 4-5-1, 6-1-3, and HBJ-127 are described in Critical Reviews in Immunology (2000); 20: 167-196.

### 7) Analysis by FACScan:

Three kinds of antibodies (namely, mouse anti-human monoclonal antibodies 4-5-1, 6-1-3, and HBJ-127) are put into tubes for FACScan in an amount of 100 μl, to which test cells were added in the same amount. As a control, PBS (Ca⁺⁺) was prepared. The tubes were left stand still at 37° C. for 11 minutes and then at 4° C. for 20 minutes, centrifugation was performed at 1500 rpm for 5 minutes, the supernatant was thrown away, PBS (Ca⁺⁺) was added in an amount of 4 ml, centrifugation was performed at 1500 rpm for 5 minutes, and the supernatant was thrown away. To the tubes were added secondary antibodies, FITC-goat IgG (anti-mouse whole IgG, Cappel Co., Cat #. 55493) in an amount of 50 μl, which were left stand still at 4° C. for 30 minutes. Again, PBS (Ca⁺⁺) was added in an amount of 4 ml, which was centrifuged at 1500 rpm for 5 minutes, the supernatant was removed therefrom, the residue was stirred, to which a 1% formalin-PBS solution was added in an amount of 300 μl to 500 μl for suspension. About 10,000 cells were measured. The migration rate thereof was compared to that of the monocyte macrophage fraction, and when there was a difference of 10% or more, it was regarded as significant.

### (Results)

The results are as follows in Table 1.

TABLE 1

| | | Migration of | | |
|---|---|---|---|---|
| | | FRP-1/CD98/4F2hc | | |
| | | expression of macrophage | | |
| | | fraction by FACScan | | |
| Agonist | | (migration rate %) | | |
| addition at the | Test compound | Monoclonal antibodies | | |
| time when | (final | against FRP-1/CD98/4F2hc | | |
| starting human | concentration: | FRP-1 | | CD98 |
| PBMC culture | nM) | 4-5-1 | 6-1-3 | HBJ-127 |
| Human PBMC + LPS | Solvent | 1.6 | 2.7 | 1.2 |
| (final | (control) | | | |
| concentration: | Compound (6-2) | 32.7 | 29.8 | 11.0 |
| 80 μg/ml) | (10 nM) | | | |
| | Compound (7-3) | 2.6 | 2.0 | 5.4 |
| | (100 nM) | | | |

### (Discussion)

Table 1 shows that the compound (6-2) activated the molecules against the FRP-1/CD98/4F2hc of the amino acid transporter expressed on the macrophage fraction of the human PBMC, which was stimulated and cultured with LPS, and increased the expression thereof. On the other hand, the compound (7-3) did not increase the expression.

Although not shown in the Table, when a different normal human PBMC was used, the compound (6-2) showed an action of selectively lowering the expression to 5% or less in the case where the expression of the molecule was enhanced to 90% or more.

### Experimental Example 2

### Experimental Procedure

In the same manner as in 1) to 5) of Experimental Example 1, to human peripheral blood mononuclear cells, the test compound (6-2) (final concentration: 10 nM) or the test compound (7-3) (final concentration: 100 nM) was added at the same time when LPS (final concentration: 80 μg/ml) was added, followed by 6-day culture. Further, in the same manner as above, using rabbit anti-human 4F2hc polyclonal antibodies (see Biol Pharm Bull 30:415-422, 2007) as a primary antibody, the peak value of HAT expressed on the fraction of large-macrophages (larger than monocyte macrophages) in human PBMC was measured by FACScan using a commercially available cell fix/cell membrane infiltration kit (trade name: BD Cytofix/Cytoperm™ Kit, catalog No. 554714, BD Biosciences).

As a control, the one with no test-compound addition was used, and as a rabbit anti-human rBAT (related to b^{0,+}-type amino acid transporter) polyclonal antibody, the one obtained by coupling a synthesized peptide composed of an amino-terminal of human rBAT (MAEDKSKRDSIEMSMKGC (SEQ ID NO: 2)) and a carboxyl-terminal of mouse b^{0,+}AT (BAT1) (CHLQMLEWPEKDPE (SEQ ID NO: 3)) to a carrier KLH (keyhole-limpet hemocyanin), and immunizing a rabbit with it.

### (Results)

The results are shown as follows in Table 2.

TABLE 2

| | | Change in HAT expressed on | |
|---|---|---|---|
| | | large-monocyte fraction of | |
| | | human PBMC after addition | |
| Addition of | Test compound | of macrophage-activating | |
| macrophage-acti- | (final | substance and culture | |
| vating substance | concentration | (peak value by FACScan) | |
| to human PBMC | nM) | 4F2hc | rBAT |
| Human PBMC + | Solvent | 23 | 37 |
| LPS (final | (control) | | |
| concentration: | Compound (6-2) | 10 | 40 |
| 80 μg/ml) | (10 nM) | | |
| | Compound (7-3) | 20 | 38 |
| | (100 nM) | | |

### (Discussion)

Table 2 shows that the test compound (6-2) selectively decreased the amount of 4F2hc in the heavy chain of HAT without any influence on the amount of rBAT, but the test compound (7-3) did not show any effects. That is, it was confirmed that the test compound (6-2) has an effect of ameliorating glomerular lesions. Therefore, it can be said that by selecting a compound which has an effect of regulating the amount of 4F2hc expression in a similar way, the compound capable of ameliorating glomerular lesions can be screened.

### Experimental Example 3

### Experimental Procedure

The same procedures as in Experimental Example 2 were taken except the following conditions: human leukemia culture cell line THP-1 was used instead of the human peripheral blood mononuclear cells; the final addition concentration of the test compound (6-2) was set to 100 nM, and the final addition concentration of the test compound (7-3) was set to 1000 nM; and fetal bovine serum was used instead of the human AB serum. The peak value of HAT expressed on the fraction of large-cells in human leukemia culture cell strain THP-1, compared with those not stimulated with LPS, was measured. As a control, the one with no test-compound addition was used, and the peak value of HAT was measured by FACScan using a commercially available cell fix/cell membrane infiltration kit (trade name: BD Cytofix/Cytopem™ Kit, catalog No. 554714, BD Bioscience).

### (Results)

The results are shown as follows in Table 3.

TABLE 3

| | | Change in HAT expressed on |
|---|---|---|
| | | large-cell fraction of |
| | | THP-1 after addition of |
| Addition of | Test compound | macrophage-activating |
| macrophage-acti- | (final | substance and culture |
| vating substance | concentration | (peak value by FACScan) |
| to THP-1 | nM) | 4F2hc |
| THP-1 + LPS (final | Solvent | 12 |
| concentration: | (control) | |
| 80 μg/ml) | Compound (6-2) | 25 |
| | (100 nM) | |
| | Compound (7-3) | 11 |
| | (1000 nM) | |

### (Discussion)

Table 3 shows that the test compound (6-2) remarkably changed the amount of 4F2hc in the heavy chain of HAT, whereas the test compound (7-3) did not make any change. That is, it is confirmed that the test compound (6-2) has an effect of ameliorating glomerular lesions. Therefore, it can be said that by selecting a compound which has an effect of regulating the amount of 4F2hc expression in a similar way, using the human leukemia culture cell line THP-1, the compound capable of ameliorating the glomerular lesions can be easily screened.

### Experimental Example 4

### Experimental Procedure

The same procedures as in Experimental Example 2 were taken except the following conditions: human leukemia culture cell line THP-1 was used instead of the human peripheral blood mononuclear cells; the final addition concentration of the test compound (6-2) was set to 100 nM; fetal bovine serum was used instead of the human AB serum; and human recombinant interferon γ (PHP050, Serotec) (final concentration: 100 ng/ml) was used instead of LPS. Also, using rabbit anti-human 4F2hc polyclonal antibodies (see Biol Pharm Bull 30: 415-422, 2007) as the primary antibody, the peak value of HAT expressed on the fraction of large-cells in human leukemia culture cell strain THP-1, compared with those not stimulated with LPS, was measured. As a control, the one with no test-compound (DMSO alone) was used, and the rabbit anti-human rBAT polyclonal antibodies were obtained in the same manner as in Experimental Example 2.

### (Results)

The results are shown as follows in Table 4.

TABLE 4

| | | Change in HAT expressed on | |
|---|---|---|---|
| | | large-cell fraction | |
| | | of THP-1 after addition | |
| Addition of | Test compound | of macrophage-activating . . . | |
| macrophage-acti- | (final | substance and culture | |
| vating substance | concentration | (peak value by FACScan) | |
| to THP-1 | nM) | 4F2hc | rBAT |
| THP-1 + human | Solvent | 16 | 55 |
| recombinant | (control) | | |
| interferon γ | Compound (6-2) | 10 | 50 |
| (final | (100 nM) | | |
| concentration: | | | |
| 100 ng/ml) | | | |

### (Discussion)

Table 4 shows that the test compound (6-2) selectively decreased the amount of 4F2hc in the heavy chain of HAT without any influence on the amount of rBAT. That is, it was confirmed that the test compound (6-2) has an effect of reducing glomerular lesions. Therefore, it can be said that by selecting a compound which has an effect of regulating the amount of 4F2hc expression in a similar way, the compound capable of reducing glomerular lesions can be screened.

### Experimental Example 5

### Experimental Procedure

### 1) Production of Rat Unilateral Ureteral Obstruction Release Model:

An experimental model was produced in accordance with the method devised and established by Ishibashi (Michio Ishibasi et al., Japanese Journal of Nephrology 42: 248, 2000), using a 8 to 9-week-old male SD rat weighing about 280 g. That is, the abdomen of the rat was opened under ether anesthesia, and its ureter was ligated at a height of inferior pole of the left kidney with a 7-0 nylon suture, and then the abdomen was closed. On day 14 after obstruction, the obstruction was released, and the urinary tract was reconstructed using a cuff. That is, 14 days later, the ligated ureteral obstruction part was partially removed, and a 25 gauge polyethylene tube (made by Nippon Sherwood) as a cuff was inserted from the lower normal ureteral stump and placed in the lumen. Next, the cuff was placed also in the expanded upper ureter, and both parts were ligated for fixation with a 7-0 nylon suture to reconstruct the urinary tract. At the same time, the right kidney was removed. The weight was measured after obstruction release, and the blood was collected on days 2, 5, and 7 after release for serum creatinine determination. On day 7, the rat was sacrificed under anesthesia and the left obstruction release kidney was removed. The weight of the removed kidney was measured, and the kidney pathomorphological examination was performed. In this model, when the test compound was not administered, the longitudinal pathomorphological study during obstruction and after obstruction release showed kidney structural disorganization, glomerular Bowman's capsule wall hypertrophy, mesangium cell hyperplasia, glomerulosclerosis, atrophied or expanded tubule, cellular infiltration into interstitium, and fibrosis.

### 2) Administration of Test Compound:

The biological effects of the test compound were studied in vivo using the model obtained in the above procedure 1). The test compound in the form of bulk powder was dissolved in sterilized physiologic saline together with gum arabic, where the concentration of the gum arabic was adjusted to 5% and the concentration of the test compound was adjusted to 30 mg/ml. The amount of 30 mg/kg was subcutaneously administered every day. Daily administration was continued for 21 days during 14 days of obstruction and 7 days after obstruction release. For controlled comparison, the solvent of 5% gum arabic was administered alone.

### 3) Observation of Glomerular Lesion:

On day 22 from the administration of the test compound, the rat was sacrificed under anesthesia, and the left obstruction release kidney was removed and neutral formalin fixed. The fixed renal tissue embedded in paraffin was sliced into 4-micrometer-thick pieces for examination.

The tissue section was observed under a microscope in order to count the number of glomeruli and the number of glomeruli having a lesion (expansion of a tubular pole associated with swollen Bowman's capsule epithelial cells or hypertrophy of a Bowman's capsule basal membrane) in the field of view, and the number of glomeruli having the lesion (referred to as “lesioned glomerulus”) per 50 glomeruli was calculated.

As for the effectiveness to improve of glomerular lesions, when the number of lesioned glomeruli per 50 glomeruli was reduced to less than or equal to half the average number of those of the control group, it was determined as improved.

### 4) Examination of the Number of Asc-1 Positive Cells:

An affinity-purified anti-rat Asc-1 polyclonal antibody (rabbit) was prepared as follows: that is, in order to prepare an anti-Asc-1 peptide polyclonal antibody (rabbit), an oligopeptide (PSPLPITDKPLKTQC (SEQ. ID NO: 4)) corresponding to amino acid residues of 517-530 of Asc-1 was synthesized (see J Biol Chem (2000); 275: 9690-9698), keyhole limpet hemocyanine was bound to the C-terminal cysteine as an adjuvant, and a rat was immunized therewith. The serum was collected on days 7 and 14 from the immunization. The serum was concentrated by means of an adsorption-dissociation operation using an affinity column to which an oligopeptide corresponding to amino acid residues of 517-530 of Asc-1 was bound in order to obtain an affinity-purified antibody having a protein content of 0.5 mg/ml. As for immunopathological examination, paraffin rat kidney tissue diluted with PBS to the optimal concentration was used.

The immune tissue staining was performed based on the protocol of Ventana HX system bench mark (Ventana Japan K.K., Tokyo) including the automated immunostaining device manufactured by Ventana Japan K.K. After deparaffinization, the tissue section was heat-treated at 100° C. for 1 hour. Using 10% goat serum (Histfine SAB-PO® kit, Nichirei Biosciences) as a blocking reagent, the affinity-purified anti-rat Asc-1 polyclonal antibody (rabbit) was reacted with a primary antibody at the optimal concentration, 50-fold dilution, at room temperature for 1 hour. As a secondary antibody, biotin-labeled anti-rabbit IgG antibody (goat) (histfine SAB-PO® kit, Nichirei Biosciences) was reacted at room temperature for 30 minutes. For color development, Simple Stain DAB Solution (histfine SAB-PO® kit, Nichirei Biosciences) was used.

The stained cells were observed under a microscope, and the number of cells and the number of Asc-1⁺ cells in the field of view were counted for calculation of the number of Asc-1+ cells per 50 glomeruli.

### 5) Observation of Tubulointerstitial Lesions:

A tissue section obtained in the same manner as in the above-mentioned 3) was observed under a microscope, where atrophy of renal tubulus associated with dilatation of renal tubulus, thickness of tubular basement membrane, and fibrosis of tubulointerstitial tissue were observed. For ±(a very light change was observed), + (a light change was observed), ++ (a middle change was observed), and +++ (a high change was observed), 0.5, 1, 2, and 3 points were given respectively, and the total score was calculated.

As to whether glomerular lesions were improved or not, when the number of lesioned glomeruli was reduced to less than or equal to half the average number of those of the control group, it was determined as improved.

### (Results)

The results are shown as follows in Table 5 to 8.

TABLE 5

| | | Glomerular lesion | | | | | | | | |
|---|---|---|---|---|---|---|---|---|---|---|
| | | Number of lesioned | | | | | | | | |
| | | glomeruli per 50 | | | | | | | | |
| | | glomeruli | | | | | | | | |
| | | Expansion | | | | | | | | |
| | | of tubular | | | | | | | | |
| | | pole | | | | | | | | |
| | | associated | | | | Tubulointerstitial lesion | | | | |
| | | with | | | | Atrophy of | | | | |
| | | swollen | Hypertrophy | Number of | Im- | tubules | | | | |
| | | Bowman's | of Bowman's | Asc-1⁺ | provement | associated | | | | Improvement |
| | | capsule | capsule | cells | of | with | Thickness | Fibrosis of | | of tubulo- |
| | Animal | epithelial | basal | per 50 | glomerular | dilatation | of basement | interstitial | Total | interstitial |
| Compound | No. | cells | membrane | glomeruli | lesion | of tubules | membrane | tissue | score | lesion |
| Compound | 1 | 2 | 6 | 9.40 | Yes | 1 | 1 | 1 | 3 | No |
| (6-2) | 2 | 5 | 9 | 9.05 | No | 0.5 | 2 | 2 | 4.5 | No |
| | 3 | 2 | 5 | 5.12 | Yes | 1 | 1 | 1 | 3 | No |
| | 4 | 1 | 8 | 8.47 | Yes | 1 | 2 | 2 | 5 | No |
| | Average | 3 ± 2 | 7 ± 2 | 8.01 ± 1.96 | 3/4 (75%) | 0.90 ± 0.3 | 1.5 ± 0.6 | 1.5 ± 0.6 | 3.9 ± 1.0 | 0/4 (0%) |
| | Significant | p = 0.05 | p = 0.001 | p = 0.004 | | | | | No | |
| | difference | | | | | | | | significant | |
| | | | | | | | | | difference | |
| Control | 1 | 9 | 24 | 1.56 | No | 3 | 1 | 3 | 7 | No |
| (5% gum | 2 | 7 | 16 | 1.13 | No | 1 | 1 | 2 | 4 | No |
| arabic) | 3 | 13 | 17 | 0.46 | No | 0.5 | 0.5 | 1 | 2 | No |
| | 4 | 3 | 17 | 1.80 | Yes | 2 | 1 | 3 | 6 | No |
| | Average | 8 ± 4 | 19 ± 4 | 1.24 ± 0.56 | 1/4 (25%) | 1.6 ± 1.1 | 0.9 ± 0.3 | 2.3 ± 1.0 | 4.8 ± 2.2 | 0/4 (0%) |

TABLE 6

Glomerular lesion

| | | Number of lesioned | | | | | | | | |
|---|---|---|---|---|---|---|---|---|---|---|
| | | glomeruli per 50 | | | | | | | | |
| | | glomeruli | | | | | | | | |
| | | Expansion | | | | | | | | |
| | | of tubular | | | | | | | | |
| | | pole | | | | | | | | |
| | | associated | | | | Tubulointerstitial lesion | | | | |
| | | with | Hyper- | | | Atrophy of | | | | |
| | | swollen | trophy of | | | tubules | | Fibrosis of | | |
| | | Bowman's | Bowman's | Number of | Improvement | associated | | uriniferous | | Improve- |
| | | capsule | capsule | Asc-1⁺ cells | of | with | Thickness | tubule | | ment of |
| | Animal | epithelial | basal | per 50 | glomerular. | dilatation | of basement | interstitial | Total | glomerular |
| Compound | No. | cells | membrane | glomeruli | lesion | of tubules | membrane | tissue | score | lesion |
| Compound | 1 | 9 | 20 | 1.35 | No | 0.5 | 0.5 | 0.5 | 1.5 | Yes |
| (7-3) | 2 | 1 | 10 | 0.47 | Yes | 0 | 0 | 0.5 | 0.5 | Yes |
| | 3 | 6 | 16 | 1.27 | No | 0.5 | 0.5 | 0.5 | 1.5 | Yes |
| | 4 | 4 | 11 | 0.53 | Yes | 1 | 0.5 | 0.5 | 2 | No |
| | Average +/− | 5 ± 3 | 14 ± 5 | 0.90 ± 0.47 | 2/4 (50%) | 0.50 ± 0.4 | 0.4 ± 0.3 | 0.5 ± 0 | 1.4 ± 0.6 | 3/4 (75%) |
| | standard | | | No | | | | | p = 0.0l | |
| | deviation | | | significant | | | | | | |
| | Significant | | | difference | | | | | | |
| | difference | | | | | | | | | |
| Control | 1 | 8 | 24 | 3.64 | No | 1 | 0.5 | 0.5 | 2 | Yes |
| (5% gum | 2 | 10 | 22 | 2.27 | No | 2 | 1 | 1 | 4 | No |
| arabic) | 3 | 9 | 20 | 1.88 | No | 3 | 1 | 0.5 | 4.5 | No |
| | 4 | 14 | 28 | -1.41 | No | 1 | 2 | 0.5 | 3.5 | No |
| | Average +/− | 10 ± 3 | 24 ± 3 | 2.30 ± 0.96 | 0/4 (0%) | 1.8 ± 1.0 | 1.1 ± 0.6 | 0.6 ± 0.3 | 3.5 ± 1.1 | 1/4 (25%) |
| | standard | | | | | | | | | |
| | deviation | | | | | | | | | |

TABLE 7

| | | Glomerular lesion | | | | | | | | |
|---|---|---|---|---|---|---|---|---|---|---|
| | | Number of lesioned | | | | | | | | |
| | | glomeruli per 50 | | | | | | | | |
| | | glomeruli | | | | | | | | |
| | | Expansion | | | | | | | | |
| | | of tubular | | | | | | | | |
| | | pole | | | | | | | | |
| | | associated | | | | Tubulointerstitial lesion | | | | |
| | | with | Hyper- | | | Atrophy of | | | | |
| | | swollen | trophy of | | Im- | tubules | | | | |
| | | Bowman's | Bowman's | Number of | provement | associated | | | | Improvement |
| | | capsule | capsule | Asc-1⁺ cells | of | with | Thickness | Fibrosis of | | of tubulo- |
| | Animal | epithelial | basal | per 50 | glomerular | dilatation | of basement | interstitial | Total | interstitial |
| Compound | No. | cells | membrane | glomeruli | lesion | of tubules | membrane | tissue | score | lesion |
| Compound | 1 | 10 | 6 | 9.48 | Yes | 3 | 2 | 2 | 7 | No |
| (3-2) | 2 | 6 | 5 | 9.55 | Yes | 3 | 2 | 3 | 8 | No |
| | 3 | 6 | 8 | 14.91 | Yes | 1 | 2 | 2 | 5 | No |
| | 4 | 5 | 7 | 6.84 | Yes | 3 | 3 | 2 | 8 | No |
| | 5 | 3 | 5 | 14.52 | Yes | 3 | 2 | 2 | 7 | No |
| | Average | 6 ± 3 | 6 ± 1 | 11.06 ± 3.51 | 5/5 (100%) | 2.6 ± 0.9 | 2.2 ± 0.4 | 2.2 ± 0.4 | 7.0 ± 1.2 | 0/5(0%) |
| | Significant | p = 0.02 | p = 0.001 | p = 0.003 | | | | | no | |
| | difference | | | | | | | | significant | |
| | | | | | | | | | difference | |
| Control | 1 | 23 | 21 | 1.45 | No | 2 | 2 | 2 | 6 | No |
| (5% gum | 2 | 22 | 18 | 1.13 | No | 3 | 2 | 3 | 8 | No |
| arabic) | 3 | 24 | 20 | 1.21 | No | 2 | 2 | 2 | 6 | No |
| | Average | 23 ± 1 | 20 ± 2 | 1.26 ± 0.17 | 0/3 (0%) | 2.3 ± 0.6 | 2.0 ± 0.0 | 2.3 ± 0.6 | 6.7 ± 1.2 | 0/3 (0%) |

TABLE 8

| | | Glomerular lesion | | | | | | | | |
|---|---|---|---|---|---|---|---|---|---|---|
| | | Number of lesioned | | | | | | | | |
| | | glomeruli per 50 | | | | | | | | |
| | | glomeruli | | | | | | | | |
| | | Expansion | | | | | | | | |
| | | of tubular | | | | | | | | |
| | | pole | | | | | | | | |
| | | associated | | | | Tubulointerstitial lesion | | | | |
| | | with | Hyper- | | | Atrophy of | | | | |
| | | swollen | trophy of | | Im- | tubules | | | | |
| | | Bowman's | Bowman's | Number of | provement | associated | | | | Improvement |
| | | capsule | capsule | Asc-1⁺ cells | of | with | Thickness | Fibrosis of | | of tubulo- |
| | Animal | epithelial | basal | per 50 | glomerular | dilatation | of basement | interstitial | Total | interstitial |
| Compound | No. | cells | membrane | glomeruli | lesion | of tubules | membrane | tissue | score | lesion |
| Compound | 1 | 16 | 29 | 1.81 | No | 1 | 1 | 2 | 4.0 | No |
| (4-2) | 2 | 6 | 9 | 7.39 | Yes | 1 | 0.5 | 2 | 3.5 | Yes |
| | 3 | 16 | 20 | 5.83 | No | 1 | 1 | 1 | 3.0 | Yes |
| | 4 | 7 | 13 | 1.10 | Yes | 1 | 1 | 1 | 3.0 | Yes |
| | Average | 11 ± 6 | 18 ± 9 | 4.03 ± 3.06 | 2/4 (50%) | 1.0 ± 0 | 0.9 ± 0.3 | 1.5 ± 0.6 | 3.4 ± 0.5 | 3/4 (75%) |
| | Significant | no | no | no | | | | | p = 0.001 | |
| | difference | significant | significant | significant | | | | | | |
| | | difference | difference | difference | | | | | | |
| Control | 1 | 25 | 17 | 12.98 | No | 3 | 2 | 3 | 8.0 | No |
| (5% gum | 2 | 14 | 22 | 14.41 | No | 3 | 2 | 3 | 8.0 | No |
| arabic) | 3 | 22 | 21 | 14.80 | No | 3 | 3 | 2 | 8.0 | No |
| | 4 | 14 | 17 | 3.09 | No | 2 | 2 | 3 | 7.0 | No |
| | Average | 19 ± 6 | 19 ± 3 | 11.32 ± 5.54 | 0/4 (0%) | 2.8 ± 0.5 | 2.3 ± 0.5 | 2.8 ± 0.5 | 7.8 ± 0.5 | 0/4 (0%) |

### (Discussion)

In the rat unilateral ureter obstruction release model, as glomerular lesions, the expansion of the glomerular tubular pole associated with the swollen Bowman's capsule epithelial cells and the hypertrophy of the Bowman's capsule basement membrane are observed. On the other hand, tubulointerstitial lesions include the atrophy of tubules associated with the dilatation of tubules, the fibrosis of interstitial tissue, and the thickness of the basement membrane. Table 5 shows that, in the group to which the compound (6-2) was administered, the uriniferous tubule lesion was not reduced but the glomerular lesion was selectively reduced.

Also, Asc-1 is one of the amino acid transporters (HATs) with H chain having FRP-1/CD98/4F2hc in common. In the group to which the compound (6-2) was administered, Asc-1 positive cells accumulated on damaged areas of the glomerular loop or the Bowman's capsule wall, and increased more significantly than in the control group. That is, the compound (6-2) activated HAT by acting on the H chain of HAT, and caused cells to express Asc-1 having an affinity for glomerular lesions. As the 4F2hc/FRP-1/CD98 in the H chain of the Asc-1 is related to increasing the adhesion of integrin and has a cell fusion activity, it might be assumed that Asc-1 positive cells were guided to the glomerular lesion site, and then adhered to and fused with the site, leading to the reduction of the glomerular lesions by the action of HAT to promote protein synthesis.

On the other hand, Table 6 shows the results of administrating the compound (7-3) to a similar model. The amelioration of tubulointerstitial lesions was seen in one rat out of four in the control group whereas the reduction was seen in all of four rats in the compound (7-3) group. These results show that the compound has an action of selectively inhibiting tubulointerstitial lesions. As the tubulointerstitial lesion was primary and the glomerular lesion was secondary in this model, the reduction of the glomerular lesion might be caused secondarily by the reduction of the tubulointerstitial lesion. In the group to which the compound (7-3) was administered, the action on the FRP-1/CD98/4F2hc in the H chain of HAT was not shown, and there were only a small number of Asc-1 positive cells in the glomeruli.

Table 7 shows analysis results of administration of the known compound (3-2) having the same action as that of the compound (6-2) to the model as above. From Table 4, it was found that the compound (3-2) lead to the same results as obtained with the compound (6-2).

Table 8 shows analysis results, for comparison, of a known compound (4-2) having the same action as in the compound (7-3). From Table 8, it was found that the compound (4-2) lead to the same results as obtained with the compound (7-3).

From all the results as described above, it was confirmed that, when a compound controlling FRP-1/CD98/4F2hc, the H chain of HAT, is identified in an in vitro screening, the compound shows pharmacological action of selectively reducing glomerular lesions or disease of kidney by allowing Asc-1 positive cells of HAT to accumulate on glomerular lesions and adhere to and fuse with the lesions, thereby facilitating a protein synthesis.

### Experimental Example 6

### Experimental Procedure

It is known that a single intravenous dose of 50 mg/kg of puromycin to a 5-week-old SD (Sprague-Dawley) male rat damages the glomerular epithelial cells, causing severe proteinuria peaking from day 6 to day 14 and gradually decreasing thereafter, and again showing tubular proteinuria (see J. R. Diamond, and M. J. Karnovsky: Focal and segmental glomerulosclerosis following a single intravenous dose of puromycin aminonucleoside, Am. J. Pathol; 1986; 122; 481-487) (hereinafter, this model will be referred to as puromycin-induced acute nephrosis model).

Using the above puromycin-induced acute nephrosis model as a model of glomerular lesions, the combinational effect of interferon γ and the composition for preventing, ameliorating, or treating glomerular lesions or disease of kidney was examined.

To a 5-week-old SD (Sprague-Dawley) male rat, 30 mg/kg/day of said composition (6-2) was subcutaneously administered every day from five days before the administration of puromycin, and at the same time, 0.25 ml (2.3×10⁴U) of rat recombinant interferon γ (by PeproTeck, final concentration: 9.09 μg/ml) was intraperitoneally administered on alternate days from five days before the administration of puromycin, followed by a single intravenous dose of 50 mg/kg of puromycin. Even after the administration of puromycin, the same amount of the composition (6-2) and the rat recombinant interferon γ were continuously administered every day and on alternate days respectively.

On days 6, 12, and 19 after the administration of puromycin, 24-hour urine collection was performed to measure the amount of rat urinary albumin excretion by ELIZA method. At the same time, the urinary creatinine concentration was also measured by enzyme method. As indexes of glomerular epithelial damage, albumin excretion and the ratio of urinary albumin excretion to urinary creatinine were compared.

### (Results)

The results are shown as follows in Table 9 and 10.

TABLE 9

| | | Albumin Excretion | | |
|---|---|---|---|---|
| | Animal | (mg/day) | | |
| Compound | No. | Day 6 | Day 12 | Day 19 |
| Compound (6-2) | 1 | 80.7 | 136.0 | 73.4 |
| alone | 2 | 115.9 | 229.1 | 128.6 |
| | Average | 98.3 | 182.6 | 101.0 |
| Compound (6-2) + | 3 | 58.0 | 105.8 | 47.6 |
| Rat recombinant | 4 | 32.4 | 128.7 | 46.7 |
| interferon γ | Average | 45.2 | 117.3 | 47.2 |

TABLE 10

| | | Urinary Albumin Excretion/ | | |
|---|---|---|---|---|
| | | Urinary Creatinine | | |
| | Animal | (1/Cr) | | |
| Compound | No. | Day 6 | Day 12 | Day 19 |
| Compound (6-2) | 1 | 12.1 | 22.3 | 11.0 |
| alone | 2 | 21.2 | 35.8 | 20.5 |
| | Average | 16.7 | 29.1 | 15.8 |
| Compound (6-2) + | 3 | 8.5 | 14.0 | 2.2 |
| Rat recombinant | 4 | 4.9 | 19.5 | 5.8 |
| interferon γ | Average | 6.7 | 16.8 | 4.0 |

### (Discussion)

The urinary albumin excretion was significantly reduced and the “urinary albumin excretion/urinary creatinine” was significantly improved in the group administered the composition (6-2) and rat recombinant interferon γ concurrently, compared with the composition (6-2) single administration group. Consequently, it was confirmed that coadministration of interferon γ provides an effective therapeutic benefit for glomerular lesions or disease of kidney.

### INDUSTRIAL APPLICATION

According to the method of the present invention, drugs for treating renal glomerular lesions can be easily screened, and therefore the method is useful in the pharmaceutical industry.

1. A method for screening a compound for preventing, ameliorating or treating glomerular lesions or disease of kidney, characterized by assaying the regulating activity of compound on 4F2hc expression shown by contacting human peripheral blood mononuclear cells or monocytes, or an established line of human cultured cells having a nature of human macrophages with a macrophage-activating substances.

2. The screening method of claim 1, wherein the assay of regulating activity of compound on 4F2hc expression shown by the test compound is performed in a manner in which the human peripheral blood mononuclear cells or monocytes, or the established line of human cultured cells having a nature of human macrophages is cultured in the presence of the macrophage-activating substances and the test compound, and the amount of 4F2hc expressed on the human mononuclear cells, or monocytes or the established line of the human cultured cells is compared to the amount of 4F2hc expressed when they are cultured in the absence of the test compound.

3. The screening method of claim 2, wherein the human peripheral blood mononuclear cells or monocytes are cultured in the presence of human AB serum, or the established line of human cultured cells having a nature of human macrophages are cultured in the presence of fetal bovine serum.

4. The screening method of claim 2, wherein the amount of 4F2hc expressed on the human mononuclear cell or monocytes or the established line of human cultured cells having a nature of human macrophages is calculated by FACScan analysis.

5. The screening method of claim 1, wherein the macrophage-activating substance is interferon or lipopolysaccharide.

6. The screening method of claim 1, wherein the established line of human cultured cells having a nature of human macrophages is Human leukemia culture cell line THP-1.

7. A method for screening a compound capable of preventing, ameliorating or treating glomerular lesions or disease of kidney comprising the steps of: (1) culturing human peripheral blood mononuclear cells in the presence of lipopolysaccharides and a test compound in human AB serum; (2) recovering the human peripheral blood mononuclear cells from the resulting culture fluid; (3) reacting antibodies of 4F2hc with the recovered human peripheral blood mononuclear cells or monocytes; (4) calculating the amount of 4F2hc by FACScan analysis; and (5) comparing the amount of 4F2hc with the amount of 4F2hc cultured in the absence of the test compound to detect increase or decrease of the amount of 4F2hc.

8. A method for screening a compound capable of preventing, ameliorating or treating glomerular lesions or disease of kidney comprising the steps of: (1) culturing an established line of human cultured cells having a nature of human macrophages in the presence of (i) lipopolysaccharides or interferon and (ii) a test compound in fetal bovine serum; (2) recovering the established line of human cultured cells having a nature of human macrophages from the resulting culture fluid; (3) reacting antibodies of 4F2hc with the recovered established line of human cultured cells; (4) calculating the amount of 4F2hc by FACScan analysis; and (5) comparing the amount of 4F2hc with the amount of 4F2hc cultured in the absence of the test compound to detect increase or decrease of the amount of 4F2hc.

9. A pharmaceutical composition which prevents, ameliorates or treats glomerular lesions or disease of kidney, containing a combination of (i) a compound having an action of regulating the expression of 4F2hc caused by contacting human peripheral blood mononuclear cells or monocytes or an established line of human cultured cells having a nature of human monocytes or human macrophages with a macrophage-activating substance and (ii) interferon.

10. The pharmaceutical composition of claim 9, wherein the compound is a compound selected by a screening method of assaying the regulating activity of compound on 4F2hc expression shown by contacting human peripheral blood mononuclear cells or monocytes, or an established line of human cultured cells having a nature of human macrophages with a macrophage-activating substances.

11. The pharmaceutical composition of claim 9, wherein the compound is 2-fluoro-5-oxotetrahydrofuran-2-carboxylic benzyl ester or 1-(4-fluorophenoxy)-3-oxo-1,3-dihydro-isobenzofuran-1-carboxylic acid.

12. The pharmaceutical composition of claim 9, wherein the pharmaceutical composition which prevents, ameliorates or treats glomerular lesions or disease of kidney is a combination drug.

13. The pharmaceutical composition of claim 9, wherein the pharmaceutical composition which prevents, ameliorates or treats glomerular lesions or disease of kidney is a kit comprising (i) a medicinal agent containing a compound having an action of regulating the expression of 4F2hc caused by contacting human peripheral blood mononuclear cells or monocytes or an established line of human cultured cells having a nature of human macrophages with a macrophage-activating substance and (ii) a medicinal agent containing interferon.

14. A method for preventing, ameliorating or treating glomerular lesions or disease of kidney including the step of administrating (i) a compound which prevents, ameliorates or treats glomerular lesions or disease of kidney and which is selected by the screening method of claim 1 and (ii) interferon to a patient in need of such prevention, amelioration or treatment of glomerular lesions or disease of kidney.
